(19) United States
(12) Patent Application Publication
(10) Pub. No.: US 2024/0330663 A1
(43) Pub. Date: Oct. 3, 2024

(54) DISTORTION COMPENSATION SYSTEM

(71) Applicant: Northern Digital Inc., Waterloo (CA)

(72) Inventors: Mark Robert Schneider, Williston, VT (US); Syamprasad Karyattuparambil Rajagopalan, Burlington, VT (US)

(21) Appl. No.: 18/618,108

(22) Filed: Mar. 27, 2024

Related U.S. Application Data
(60) Provisional application No. 63/493,887, filed on Apr. 3, 2023.

(51) Int. Cl. G06N 3/0499; G06N 3/084

(52) U.S. Cl. CPC G06N 3/0499; G06N 3/084

(57) ABSTRACT

A computer-implemented method includes receiving a first set of inductance data and a second set of inductance data, each representing one or more inductance measurements when a distorter is absent and when the distorter is present, respectively. The method includes training a machine learning system for compensating for distortion, in which the machine learning system is configured to generate an estimated value of distortion indicating an amount of distortion present in the electromagnetic field. The method includes receiving a set of inductance data and generating, by the trained machine learning system, the estimated value of distortion using the additional set of inductance data and measured pose data that represents position and orientation information for one or more sensors. The method includes providing the estimated value of the distortion for application to a computing device for calibrating the one or more sensors by compensating for the estimated value of the distortion.

### CLAIM OF PRIORITY

This application claims priority under 35 USC § 119 (e) to U.S. Patent Application Ser. No. 63/493,887, filed on Apr. 3, 2023, the entire contents of which are hereby incorporated by reference.

### TECHNICAL FIELD

The disclosure relates to use of magnetic fields for determining an object's location and orientation.

### BACKGROUND

Electromagnetic tracking (EMT) systems (also referred to as magnetic tracking systems) are used to aid in locating instruments and patient anatomy in medical procedures. These systems utilize a magnetic transmitter in proximity to one or more magnetic sensors. The one or more sensors can be spatially located relative to the transmitter and sense magnetic fields produced by the transmitter.

### SUMMARY

Tracking systems (e.g., EMT systems) can have distorters with varying size, geometries, material compositions, etc. in various positions and orientations under a transmitter array of a tracking system. The transmitter array generates a tracking volume to measure position and orientation of medical devices and instruments (e.g., a catheter) using sensors. Distorters can generate undesirable effects in measurement accuracy of sensors used in tracking systems, resulting in erroneous tracking of objects in the tracking volume. In addition to measuring position and orientation, sensors with a known physical relationship to the transmitter array (receivers as opposed to the sensors that are tracked during a medical procedure) can measure distortion of the transmitter signals due to a distorter. A distortion model based on artificial intelligence, e.g., using machine learning techniques, can be trained to compensate for distortion. The machine learning (ML) model can provide accurate sensor tracking data to the tracking system, thereby mitigating the distortion effects on the tracking volume.

Receiver data can be captured by switching the transmitter coil circuitry with that of typical sensor coil circuitry using switching circuitry (a transceiver). This allows for mutual and/or self-inductance measurements to be made of the transmitter array. As described in this specification, measurements for mutual and self-inductance can be used interchangeably with measured coupling/sensing. These values change due to the presence of a distorter. The same effect can also be accomplished using discrete receivers eliminating the need for switching circuitry to measure inductance. For example, receivers can be placed coaxially with the transmitter array, in the same plane, out of plane, or not parallel to the transmitter array in general. Transceiver switching circuitry may be typically used to configure coils of a transmitter array to receive instead of transmit, thereby measuring inductance. The transceiver design enables us accurately measure inductance, while the receivers can measure distortion in the tracking volume of the transmitter array. The receivers of the transmitter array may be configured to detect amounts of distortion from distorters in the tracking volume. The ML model can use transceiver and receiver detected amounts of distortion to identify distortion patterns, e.g., a signature corresponding to a particular distorter. The ML model can provide compensation for the distortion in a tracking volume of the transmitter array, to adjust estimates for position and orientation of objects in the tracking volume.

In an aspect, a computer-implemented method includes receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field. The computer-implemented method includes training a machine learning system for compensating for distortion by using the first set of inductance data and the second set of inductance data, wherein the machine learning system is configured to generate an estimated value of distortion, the estimated value of distortion indicating an amount of distortion present in the electromagnetic field. The computer-implemented method also includes receiving an additional set of inductance data representing one or more inductance measurements from the one or more transmitters, and generating, by the trained machine learning system, the estimated value of distortion in the electromagnetic field using the additional set of inductance data and measured pose data, wherein the measured pose data represents position and orientation information for one or more sensors. The computer-implemented method includes providing the estimated value of the distortion for application to a computing device for calibrating the one or more sensors by compensating for the estimated value of the distortion.

In some implementations, the computer-implemented method includes producing a compensation for at least one sensor of the one or more sensors, based on the estimated value of distortion and the measured pose data, and applying the compensation to adjust at least one of (i) a position, or (ii) an orientation, for the at least one sensor of the one or more sensors.

In some implementations, the computer-implemented method includes producing the compensation for the at least one sensor of the one or more sensors comprises determining at least one of (i) a first position, or (ii) a first orientation of the at least one sensor based on the measured pose data. The first position represents a position of the at least one sensor with the distorter being present and the first orientation represents an orientation of the at least one sensor with the distorter being present.

In some implementations, the computer-implemented method includes producing a compensation for at least one sensor of the one or more sensors comprises determining at least one of (i) a calibrated position, or (ii) a calibrated orientation, for the at least one sensor based on the estimated value of distortion and the measured pose data.

In some implementations, the computer-implemented method includes applying the compensation comprises providing a signal, wherein the signal adjusts the at least one sensor from the first position to the calibration position and the first orientation to the calibration orientation.

In some implementations, the computer-implemented method includes comparing the estimated value of distortion to a threshold value, wherein the distorter is present if the estimated value of distortion exceeds the threshold value.

In some implementations, the trained machine learning system is configured to generate a distorter output, the distorter output representing at least one of (i) a size, (ii) a type, (iii) a material composition, (iv) a magnetic field shape, or (v) a shape, of the distorter. Training the machine learning system can include executing training iterations until an output of the machine learning system satisfies a threshold error. Training the machine learning system includes performing training iterations until an output label of the machine learning system substantially matches a predefined distorter label.

In an aspect, a system includes one or more computers and one or more computer memory devices interoperably coupled with the one or more computers and having tangible, non-transitory, machine-readable media storing one or more instructions that, when executed by the one or more computers, perform one or more operations. The one or more operations include receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field. The operations also include training a machine learning system for compensating for distortion by using the first set of inductance data and the second set of inductance data, wherein the machine learning system is configured to generate an estimated value of distortion, the estimated value of distortion indicating an amount of distortion present in the electromagnetic field. The operations include receiving an additional set of inductance data representing one or more inductance measurements from the one or more transmitters and generating, by the trained machine learning system, the estimated value of distortion in the electromagnetic field using the additional set of inductance data and measured pose data, wherein the measured pose data represents position and orientation information for one or more sensors. The operations include providing the estimated value of the distortion for application to a computing device for calibrating the one or more sensors by compensating for the estimated value of the distortion.

In some implementations, the system includes producing a compensation for at least one sensor of the one or more sensors, based on the estimated value of distortion and the measured pose data, and applying the compensation to adjust at least one of (i) a position, or (ii) an orientation, for the at least one sensor of the one or more sensors.

In some implementations, the system includes producing the compensation for the at least one sensor of the one or more sensors comprises determining at least one of (i) a first position, or (ii) a first orientation of the at least one sensor based on the measured pose data. In some implementations, the first position represents a position of the at least one sensor with the distorter being present and the first orientation represents an orientation of the at least one sensor with the distorter being present.

In some implementations, the system includes producing a compensation for at least one sensor of the one or more sensors comprises determining at least one of (i) a calibrated position, or (ii) a calibrated orientation, for the at least one sensor based on the estimated value of distortion and the measured pose data. The system includes applying the compensation comprises providing a signal, wherein the signal adjusts the at least one sensor from the first position to the calibration position and the first orientation to the calibration orientation.

In some implementations, the system includes comparing the estimated value of distortion to a threshold value, wherein the distorter is present if the estimated value of distortion exceeds the threshold value.

In some implementations, the trained machine learning system is configured to generate a distorter output, the distorter output representing at least one of (i) a size, (ii) a type, (iii) a material composition, (iv) a magnetic field shape, or (v) a shape, of the distorter.

In some implementations, training the machine learning includes executing training iterations until an output of the machine learning system satisfies a threshold error.

In an aspect, a non-transitory, computer-readable medium storing one or more instructions executable by a computer system to perform operations including: receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field; training a machine learning system for compensating for distortion by using the first set of inductance data and the second set of inductance data, wherein the machine learning system is configured to generate an estimated value of distortion, the estimated value of distortion indicating an amount of distortion present in the electromagnetic field; receiving an additional set of inductance data representing one or more inductance measurements from the one or more transmitters; generating, by the trained machine learning system, the estimated value of distortion in the electromagnetic field using the additional set of inductance data and measured pose data, wherein the measured pose data represents position and orientation information for one or more sensors; and providing the estimated value of the distortion for application to a computing device for calibrating the one or more sensors by compensating for the estimated value of the distortion.

The foregoing and other advantages and features herein will, in part, appear in the following detailed description and claims, taken together with the drawings.

### DESCRIPTION OF DRAWINGS

FIG. 1 is a schematic diagram of an electromagnetic tracking system.

FIG. 2 is a schematic top view of an example of a field generator assembly.

FIG. 3 is a diagram representing the presence of an object capable of distorting measurements of a magnetic tracking system.

FIG. 4 is a diagram a magnetic tracking system with non-planar coils.

FIG. 5 is a diagram of a transmitter measuring self-inductance in the presence of a distorter.

FIG. 6 is a diagram of transmitters measuring mutual inductance in the presence of a distorter.

FIG. 7 is a diagram of a magnetic tracking system measuring inductance in the presence of a distorter.

FIG. 8 is a computational system that determines and compensates for distortion.

FIG. 9 is a flowchart of operations of a training data collector to collect training data absent distorters for training a distortion compensation machine learning system.

FIG. 10 is a flowchart of operations of a training data collector to collect training data with distorters present for training a distortion compensation machine learning system.

FIG. 11 is a data flow of operations of for distortion compensation using a machine-learning model.

FIG. 12 is a flowchart of operations of a distortion manager.

FIG. 13 illustrates an example of a computing device and a mobile computing device that can be used to implement the techniques described.

Like reference numbers and designations in the various drawings indicate like elements.

### DETAILED DESCRIPTION

Tracking systems (e.g., EMT systems) track objects such as medical devices in an environment, e.g., a surgical theater, operating room. A tracking system provides pose information (i.e., position and orientation) to locate instruments (e.g., a catheter) and acquire measurements with respect to a patient's anatomy. Medical procedures that employ the tracking systems can span many domains and can include surgical interventions, diagnostic procedures, imaging procedures, radiation treatment, etc. Distorters (e.g., objects capable of distorting an electromagnetic field) may inadvertently be present in the tracking system environment, thereby affecting measurement accuracy. The ability of a tracking system to provide information (e.g., tracking data) to a user, e.g., a surgeon, can be critical to successfully treat a patient, e.g., executing a successful surgical procedure.

Distortion present in an environment (e.g., a surgical theater) may affect the tracking accuracy of the sensors. Due to this distortion, one or more tasks may be improperly executed; for example, sensor pose may be incorrectly calculated, thereby causing inaccurate tracking of surgical instruments by the EMT system in the surgical theater.

Artificial intelligence can provide distortion compensation of sensor pose used in an EMT system. For example, distortion compensation using artificial intelligence, e.g., machine learning (ML) techniques, may perform numerous training iterations to learn patterns of distortion effects, e.g., a distorter in the environment of an EMT system. An EMT system with a ML model is able to obtain highly accurate estimations for sensor poses of the EMT system. The ML model can also enable the EMT system to estimate amounts of distortion from previously unknown types of distortions. By doing so, the EMT system may compensate for uncommon sources of distortion, and various types of distorters, regardless of distorter shape, size, type, material composition, etc.

EMT systems utilize electromagnetic fields (e.g., magnetic fields) to track objects (e.g., medical devices, supplies) in various environments. For example, an EMT system used in an operating environment may monitor and record measurements about medical tools (e.g., a catheter, biopsy needle, ultrasound probe) such as the position and orientation of the medical tool. EMT systems are incredibly useful for performing medical procedures but can be susceptible to distortion being introduced that can degrade position and orientation measurements (e.g., of medical tools). One or more sources may introduce such distortion; for example, distortion may be introduced by electromagnet field sources (e.g., drills are good magnetic field generators), other sensor technologies, objects present (e.g., needed medical tools that include conductive or ferromagnetic materials, unwanted objects that include conductive or ferromagnetic materials and that are proximate to the generated fields, etc.) To address such distortion, one or more techniques may be employed; for example, one or more calibration techniques can be used to calibrate measurements (e.g., position and orientation measurements). Due to the complex nature of some environments (e.g., surgical theaters having multiple sources of distortion with varying impact), the application of artificial intelligence e.g., machine learning techniques, can improve the accuracy of sensor position and orientation estimates and appropriately compensate EMT systems for accurately tracking medical equipment.

Many EMT systems include numerous sensors (e.g., five or six degrees of freedom sensors) to support medical procedures by identifying the respective three-dimensional locations and orientations of medical devices and equipment. For example, the EMT systems can guide medical professionals when performing procedures (e.g., in image-guided procedures) or achieve other benefits such as reducing reliance on other imaging modalities (e.g., fluoroscopy, which exposes patients to ionizing radiation that can potentially create health risks) or track medical devices and equipment (e.g., robotic arms used to assist in surgery).

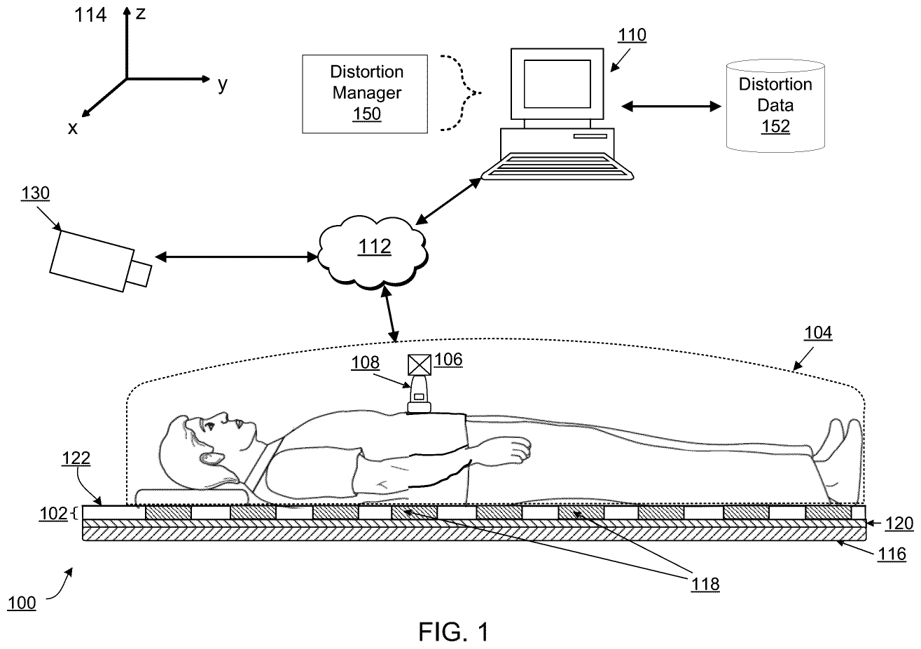

Referring to FIG. 1, a schematic diagram presents an exemplary electromagnetic tracking system 100 that includes a magnetic field generating assembly 102 configured to generate magnetic fields within a given a three dimensional region (e.g., a volume 104). A sensor assembly 106 placed on a medical device 108 within the given volume 104 detects and/or measures the magnetic fields and communicates the measurements to a computer system 110, e.g., by way of a communications link 112 (e.g., wired or wireless connections). While the medical device 108 is shown to be an ultrasound probe in FIG. 1, this is only for illustrative purposes. The medical device 108 can be any device or tool used for surgical or medical procedures, e.g., a catheter, an endoscope, a scalpel, biopsy needles, body-mounted position sensors, etc.

Based on the measurements by the sensor assembly 106, the computer system 110 can compute a position of the sensor assembly 106 (and therefore of the medical device 108) with respect to a coordinate system 114 thereby allowing motion tracking of the sensor assembly 106 (and correspondingly the medical device 108) within the region 104. This is useful in advanced surgical procedures where the sensor assembly 106 tracks the motion of the objects as a medical procedure is being executed.

Due to the sensitivity of the generated magnetic fields in the electromagnetic tracking system 100, the presence of metallic materials (e.g., ferromagnetic and/or conductive materials) in proximity to the magnetic fields can present distortion. For example, conductive materials can generate eddy currents with corresponding fields that distort the shape of the induced fields and potentially cause erroneous sensor measurements and reduce tracking accuracy. Permeable materials may also distort the magnetic fields, e.g., by bending or changing the shape of the magnetic fields. Distortions in an environment with the electromagnetic tracking system 100 can generate severe measurement errors and reduce the overall effectiveness of the electromagnetic tracking system 100. Computational solutions such as machine learning techniques may be used to compensate for the distortion caused by these materials.

As illustrated in FIG. 1, the computer system 110 executes the distortion manager 150 that uses machine learning techniques (e.g., a distortion model, also referred to as an ML model) to compensate for distortion in the electromagnetic tracking system 100. The computer system 110 can access distortion data 152, which can include training examples of collected inductance data, sensor data, and distorted pose estimations from the electromagnetic tracking system 100. The distortion data 152 can include inductance data measured from a magnetic field generated by field generating assembly 102, and sensor data collected by sensor assembly 106. The distortion data 152 also includes pose estimations (e.g., position and orientation) of objects in the electromagnetic tracking system 100 that can be distorted. Furthermore, these training examples can be generated and stored for training an ML model (e.g., for historical training, real-time training) of the distortion manager 150. The distortion manager 150 learns relationships between the different sets of data to compensate and accurately estimate poses of objects, e.g., accounting for distortion in the tracking system 100. The distortion manager 152 can collect additional inductance data, sensor data, and pose estimates (e.g., including distorted measurements) to perform real-time distortion compensation when tracking objects in the tracking system 100. In other words, the distortion manager 150 may be used by the computer system 110 to compensate for the effects of distortion and to determine an accurate position and orientation of a sensor.

A model using the machine learning techniques described herein can compensate for the distortive effects of a distorter. The ML model can compensate for distortive effects of the distorter without classifying a type, shape, size, or other physical characteristics of the distorter. For example, the ML model can determine an amount of magnetic field distortion generated by the distorter, and compensate tracking estimates of objects in the electromagnetic tracking system 100. The ML model uses the determined amount of magnetic field distortion to improve accuracy and tracking of the true position and orientations of an object in the electromagnetic tracking system 100. By determining an amount of distortion, (e.g., instead of classifying multiple physical characteristics related to the distorter) the ML model can efficiently compensate for distortion effects, thereby improving computational efficiency of EMT systems, e.g., the computer system 110 of the electromagnetic tracking system 100. The improved measurement accuracy provided by the ML model reduces likelihood of performing extraneous measurements in the presence of distortive effects. Furthermore, the improved measurement accuracy provides a reduction in computational demands during operation of the electromagnetic tracking system 100, e.g., capturing sensor measurements, by providing high fidelity data that is compensated for distortion. The improved data collection for the electromagnetic tracking system 100 improves operability and reduces computational demand through the achieved improvement in measurement accuracy.

The computer system 110 may include one or more processors to perform the functions of the distortion manager 150, but implemented as software, hardware, or some combination therein. The distortion manager 150 may also access distortion information across multiple distributed computing systems, e.g., over one or more networks. For example, along with identifying the presence of distorters, the distortion manager 150 may use inductance data and sensor data from multiple tracking system environments. In some implementations, the distortion manager 150 can be performed by multiple computing system devices across multiple environments that include a tracking system, e.g., electromagnetic tracking system 100.

Referring to the field generating assembly 102 of FIG. 1, in some implementations, the field generating assembly 102 is relatively thin in dimensions (e.g., height) and is mounted on a flat surface such as a surgical table 116. Although FIG. 1 depicts the field generating assembly 102 mounted on the surgical table 116 (e.g., as a flat field generator), the field generating assembly 102 may be integrated into the surgical table 116 by possibly embedding the field generating assembly 102 within the surgical table 116. In this particular arrangement, the field generating assembly 102 includes multiple field generators 118 (e.g., referring to a single field generator or multiple field generators) that each may include one or more electromagnetic coils that produce a magnetic field (e.g., by passing current through each coil). Additionally, an electromagnetic tracking system 100 that uses a field generating assembly 102 that generates a magnetic field may be referred to as a magnetic tracking system 100 in some implementations.

In some implementations, distorters (composed of conductive and/or highly magnetically permeable material) may be purposefully introduced to generate a desired shape of the induced magnetic fields. For example, a plate 120 can be used to shield or shape the induced magnetic fields, although multiple plates can be used. For example, multiple plates can be positioned and orientated in any configuration of the magnetic tracking system 100, e.g., around the magnetic field, above the magnetic field, below the magnetic field. In some implementations, one or more of the multiple plates are conductive and/or have high magnetic permeability.

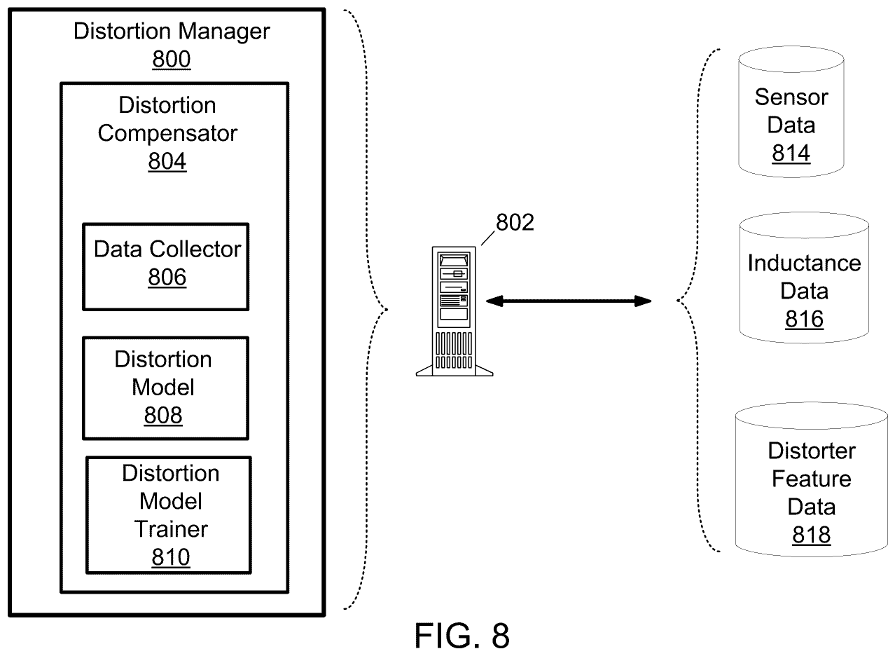

FIG. 1 illustrates the magnetic tracking system 100 in which induced magnetic fields below the plane of the field generator assembly 102 is not desirable. In such cases, the plate 120 is included to significantly attenuate the magnetic field located below the field generator assembly 102, e.g., shielding the magnetic tracking system 100 from distortion positioned below the field generator assembly 102. Consequently, in some implementations, the plate 120 distorts the magnetic fields and affects measurement accuracy of the magnetic tracking system 100 while attenuating other sources of distortion. Any distortions in the generated magnetic fields can be identified, estimated, and compensated by a distortion manager 150, as described in FIG. 8 below.

The sensor assembly 106 is used to detect the magnetic fields induced in the region 104. In some implementations, the sensor assembly 106 may include one or multiple sensors (e.g., a sensor array) that incorporate one or more types of sensing technology. For example, the sensor assembly 106 may include a coil, several coils, one or more Hall sensors, a flux gate sensor or other types of sensors capable for measuring characteristics of an electromagnetic field, (e.g., magnetic field flux, magnetic field differential.) In some implementations, magnetic fields generated by one or more field generators 118 induce electromotive forces (EMFs) in the sensor assembly 106. The measured EMFs represent the measured local values of magnetic fields at the location and orientation of the sensor assembly 106 in a three dimensional space that defines the region 104.

The sensor assembly 106 outputs signals that represent one or more measured magnetic fields corresponding to the individual fields induced by activating one or multiple field generators 118 (e.g., different sets of generators such as generator pairs). Measuring several fields induced within the region 104 allows tracking of the sensor assembly 106 with multiple degrees of freedom. For example, multiple different magnetic fields may be used to determine five degrees of freedom (x, y, z, φ, θ), where the coordinates (x, y, z) and angles (φ, θ) specify the three-dimensional location and orientation, respectively, of the sensor with respect to a reference. As another example, multiple magnetic fields can also be used to determine six degrees of freedom including x-axis position, y-axis position, z-axis position, roll, pitch, and yaw.

In some implementations, the field generator assembly 102 also includes a covering layer 122 that substantially encases the field generators 118, providing an interface surface for the patient (e.g., to sit or lie on) during a procedure. The covering layer 122 may be constructed from various types of material or material combinations, for example, a non-conductive or non-magnetic material such as plastic may be incorporated into the covering layer 122. In some implementations, the covering layer 122 is configured to provide mechanical support to the field generators 118, e.g., embedded within a solid covering layer 122. In some implementations, the covering layer 122 can simply cover the field generators 118, but can also be configured to accommodate the possible motions (e.g. translational, rotational, etc.) of the field generators 118.

In some implementations, the measured magnetic field values depend on one or more system related parameters and the three-dimensional location and the orientation of the sensor coil. The number of sets of field generators 118 and the number of sensor coils in the sensor assembly 106 may vary depending upon number of factors (e.g., gain) including the particular measurement application.

In some implementations, the magnetic tracking system 100 includes an optical system 130 to capture optical data from objects in volume 104. The optical data from the optical system 130 can be provided to computer system 110 to generate training examples for the ML model of the distortion manager 150. For example, the optical data from the optical system 130 can include position and orientation measurements of objects (e.g., sensor assembly 106) in the volume 104. In some implementations, the computer system 110 determines training examples (e.g., in the presence of the distorter, in the absence of the distorter). The optical system 130 can include one or more camera devices, light sources, detectors, or some combination therein. The camera devices of the optical system 130 can capture optical data such as images, videos, and optical measurements, e.g., through a light source and detector of the optical system 130.

In some implementations, different sets of field generators 118 may be excited at separate time instances. As an example, the computer system 110 can identify the set of field generators 118 inducing the magnetic field detected by the sensor assembly 106 based on information communicated to the computer system 110 from the field generator assembly 102. In some examples, the field generating assembly 102 may drive each set of field generators 118 at different frequencies that the computer system 110 can identify. The computer system 110 may decompose measured EMFs from the sensor assembly 106 into frequency components that match to individual sets of field generators, identifying the particular set of field generators responsible for each measured field. The sensor assembly 106 sends the measured field values to the computer system 110 that uses the measured magnetic field values to determine the pose, e.g., a position, and an orientation of the sensor assembly 106.

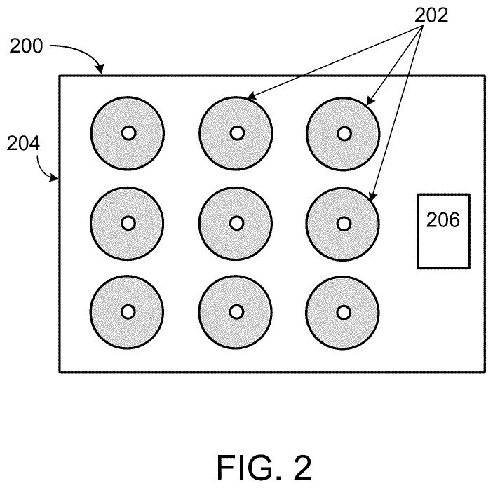

Referring to FIG. 2, a schematic diagram depicts a top view of an exemplary field generator assembly 200 that includes a plurality of individual field generators 202 (similar to the field generators 118 shown in FIG. 1) distributed on a layer 204. Even though FIG. 2 shows nine field generators 202 distributed in a particular manner, this is only for illustrative purposes and should not be considered limiting. More or fewer field generators 202 may be placed in the field generator assembly 200 in various other distributions, including distributions in multiple planes (e.g., vertically distributed planes). The layer 204 can be made of substantially the same material as the covering layer 122 described above with respect to FIG. 1. In some implementations, the layer 204 can be the top surface of a conductive or magnetically permeable plate or shield (e.g., plate 120 shown in FIG. 1).

One or more field generators 202 can be connected with one other and to a main power supply by wires (not shown). The connections can be configured in accordance with which generators are scheduled to be simultaneously activated. In some implementations, some or all of the field generators 202 can be part of the same field generator assembly 200 without being connected to each other, e.g., further enabling fields of different geometries, shapes, sizes, magnetic field strength. The field generator assembly 200 may also include a circuit board 206. In some implementations, the circuit board houses an electronic module that controls the excitation or firing of the sets of field generators 202. The circuit board 206 may also include a memory that, in communication with the computer system 110, stores configuration data associated with the field generator assembly 200. The circuit board 206 may also serve as an interface with a power supply powering the field generator assembly 200. In some implementations, the computer system 110 can be implemented as a part of the circuit board 206.

The electromagnetic coils of the field generating assembly 200 can be formed by winding a conductor, such as an electrical wire, around a core of magnetic material or a non-magnetic material (e.g., air). When a current is passed through the windings of a coil, a magnetic field is generated and extends through the center of the coil along its longitudinal axis and circles back around the outside of the loop or coil. The magnetic field circling each loop or winding of wire combines with the fields from the other loops to produce a concentrated field down the center of the coil. The strength and shape of a coil's magnetic field can be controlled, e.g., by adjusting the current, the number of loops or windings of the coil through adjustable taps, and other parameters and characteristics associated with the coils.

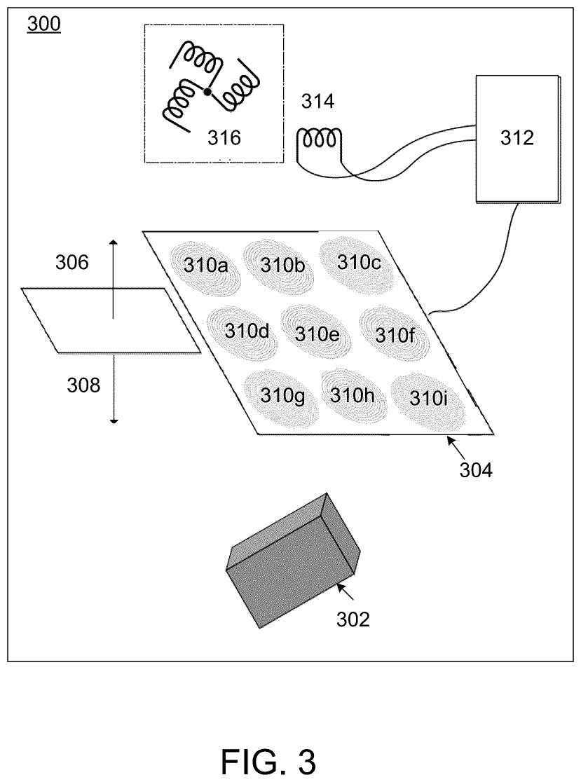

FIG. 3 illustrates an exemplary magnetic tracking system 300 in which a distorter 302 is present and can distort measurements. To address the distortion, the magnetic tracking system 300 can detect the presence of the distorter, quantify the distortion, e.g., through inductance measurements, and compensate for the effect of distortion on a tracking sensor 314 and tracking sensor 316. The magnetic tracking system 300 can be employed in the tracking system 100 (of FIG. 1) and includes a magnetic field generating assembly 304 that is configured to generate magnetic fields within a given a three dimensional region e.g., a tracking volume 306. The magnetic tracking system 300 also includes a distorter volume 308, illustrated as being opposed relative to the tracking volume 306. In some implementations, the distorter volume 308 and the tracking volume 306 can be oriented differently, i.e., relative to the magnetic tracking system 300, such as above the field generating assembly 304. In some implementations, the tracking volume 306 and distorter volume 308 can be placed in multiple locations relative to the magnetic tracking system, e.g., below the field generating assembly 304. In some implementations, there can be multiple tracking volumes 306 and/or multiple distorter volumes 308 positioned relative to the field generating assembly 304.

The distorter 302 may be located in a distortion volume 308, with respect to the magnetic tracking system 300. While graphically represented as a rectangular prism, the distorter 302 can be any shape or size. As an example, a distorter 302 can be a fixed structure such as an operating table used in a surgical environment. The distorter 302 can be a known source of distortion to the user, although in many examples, the sources of distortion from a distorter 302 are unknown. In some examples, the distorter 302 can be a medical device that changes position in the tracking volume 306, e.g., the medical device is used to perform surgery. Due to the distortive effects of the distorter 302, the measurement accuracy of tracking sensor 314 and tracking sensor 316 can be compromised.

The magnetic tracking system 300 includes an electronics unit 312 that is coupled to tracking sensor 314 (e.g., a five degree of freedom sensor) and tracking sensor 316 (e.g., a sensor with a number of non-coplanar coils). In some implementations, a tracking sensor can include any number of orthogonal and/or planar and/or concentric coils. The tracking sensor 314 and tracking sensor 316 are positioned within the tracking volume 306 such that the pose of the tracking sensor 314 and tracking sensor 316 can be estimated by magnetic tracking system 300. The distortion effects introduced by distorter 302 can compensated by a distortion manager, e.g., distortion manager 150 of FIG. 1, to improve position and orientation estimates of tracking sensor 314 and tracking sensor 316.

The electronics unit 312 can measure induced voltage across the tracking sensor 314 and tracking sensor 316, e.g., receive signals from the tracking sensors. The electronics unit 312 is also coupled to the magnetic field generating assembly 304 to control and drives the respective coils of the magnetic field generating assembly 304. In some implementations, the computer system 110 (referring to FIG. 1) can include an electronics unit 312. In some implementations, the computer system 110 can measure induced voltages across tracking sensors and/or control the magnetic field generating assembly 304 by providing drive signals.

The magnetic field generating assembly 304 includes nine field generators 310a-310i, although any number of field generators may be used. Each field generator 310a-310 may include one or more electromagnetic coils generating a magnetic field. An electronics unit 312 can provide excitation signals for the field generators 310a-310i, measure the inductance of each field generator 310a-310i, and measure the mutual inductances between each of the field generators 310a-310i. Though these inductance measurements, the distortive effects (e.g., amounts of distortion) generated by the distorter 302 can be determined and compensated to improve tracking objects in the magnetic field, e.g., magnetic tracking system 300.

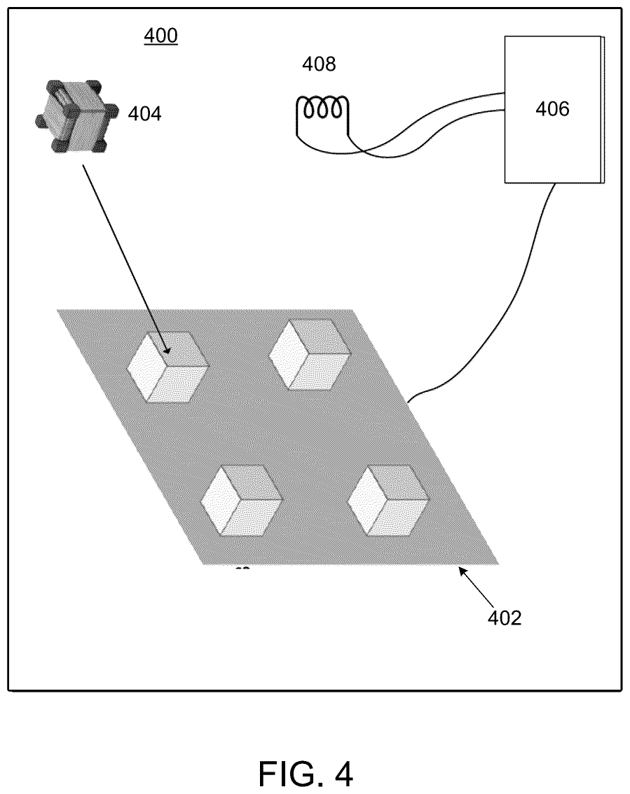

FIG. 4 illustrates another example of a magnetic tracking system 400 with field generators that are not planar, e.g., a different implementation of a field generating assembly and coils. The magnetic tracking system 400 includes a field generating assembly 402 that is configured to generate magnetic fields. The field generating assembly 402 is coupled to an electronics unit 406 similar to the electronics unit 312, illustrated in FIG. 3 above. The electronics unit 406 is further coupled to a tracking sensor 408, also similar to tracking sensor 314 illustrated above. The electronics unit 406 is configured to receive signals (e.g., measurements) from tracking sensor 408, as well as provide driving signals to the transmitters of the field generating assembly 402. The field generating assembly 402 can include a number of tracking units 404 that can be used as transmitters.

In some implementations, the tracking unit 404 has walls that are shaped into a shell around a hollow core. The walls can be a flexible circuit that is conductive and contains electronic circuitry to transmit signals. The flexible circuit is folded upon itself into a cuboid configuration to provide structural rigidity for the additional components that surround the flexible circuit. These components can include a ferromagnetic fabric material that covers the flexible circuit, which is inserted into a plastic bobbin that is wound with coil wire. The coil wire is terminated by soldering to an exposed portion of the flexible circuit. The coil drive circuitry is thereby contained within the coil wire for a compact and light unit. In some implementations, the tracking unit 404 can include concentric coils of wire.

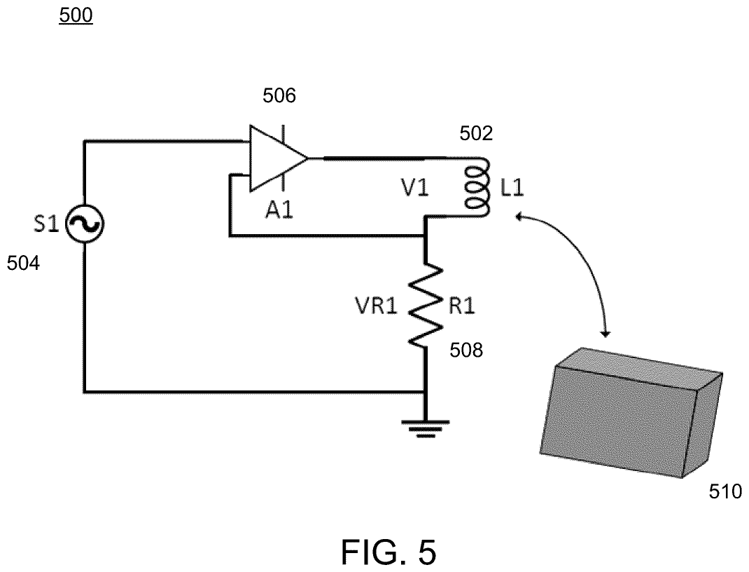

FIG. 5 is a schematic diagram illustrating exemplary self-inductance measurements in an electromagnetic tracking system in the presence of a distorter 510 (e.g., similar to distorter 302, referring to FIG. 3). Self-inductance measurements may be captured by the EMT system to identify sources of magnetic field contributions generated by a particular circuit. The field generators described above can be used to perform self-inductance measurements to determine if the distorter 510 is present. Self-inductance measurements can also be performed without the presence of the distorter 510. In a self-inductance measurement, a single field generator can be used to determine the inductance. The circuitry 500 that performs the self-inductance measurements can be included, e.g., in the electronics unit 312 of FIG. 3. The circuitry 500 can drive field generators 502 (e.g., transmitter coils, field generators) to generate electromagnetic fields. The circuitry includes waveform sources 504 (e.g., individual sources, multiplexed sources, etc.) to vary signal parameters (e.g., amplitude, phase, frequency, waveshape), amplifiers 506 to drive the field generators 502, and resistors 508 to measure the current through the field generators.

For example, the current I1(t) through the field generator 502 is described by equation (1), as a function of voltage VR1(t) and resistance R1:

$$I 1 ( t ) = \frac{VR 1 ( t )}{R 1} \qquad ( 1 )$$

The voltage V1(t) across the field generator 502 is described by equation (2) as a function of inductance L1 and current I1(t):

$$V 1 ( t ) = L 1 \frac{dI 1 ( t )}{dt} \qquad ( 2 )$$

Combining the two equations above, the inductance L1 can be represented by equation (3):

$$L 1 = V 1 ( t ) R 1 / \frac{dVR 1 ( t )}{dt} \qquad ( 3 )$$

The inductance can be determined using the voltage V1(t) across the field generator 502, the voltage VR1(t) across the resistor 508, and the resistance R1 of the resistor 508. The measurements of V1(t) and VR1(t) can be performed, e.g., by instrumentation. The instrumentation can also determine other values (e.g., derivatives, magnitudes, phases, etc.) by processing the waveforms. As discussed above, the inductance can be used by the distortion manager 150 (referring to FIG. 1) to correct for the presence of a distorter 510.

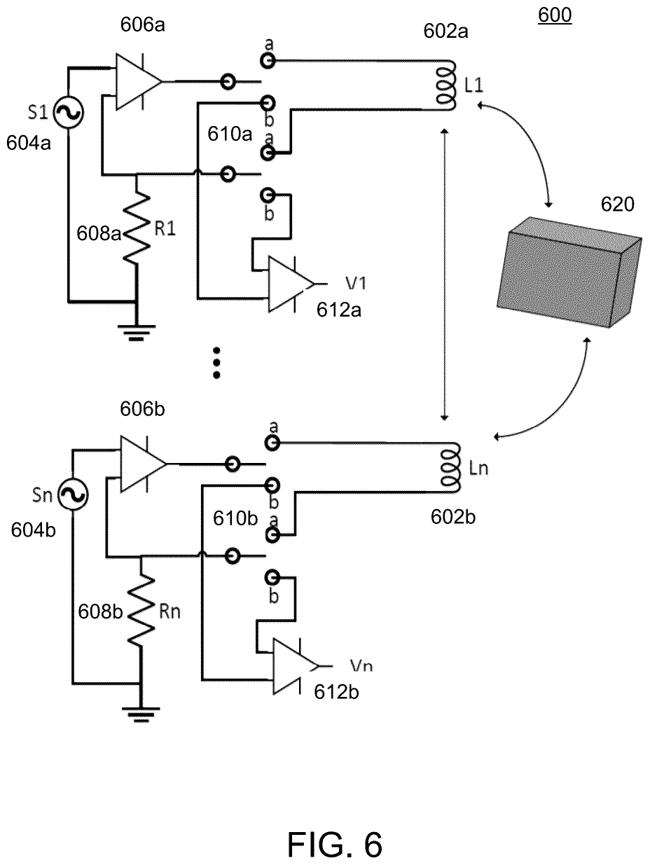

FIG. 6 is a schematic diagram illustrating exemplary mutual-inductance measurements in an electromagnetic tracking system, e.g., in the presence of a distorter 620 (e.g., similar to distorter 302, referring to FIG. 3). Mutual-inductance measurements may be captured by the electromagnetic tracking system to identify sources of magnetic field contributions generated by one circuit due to another circuit. For example, a circuit used in the EMT system may affect a magnetic field generated by a second circuit in the EMT system by generating an EMF that adjusts the voltage of the second circuit. The field generators described above can be used to perform mutual-inductance measurements to determine whether distorter 620 changes the mutual inductance. In mutual-inductance measurements, one field generator can receive signals from another field generator, and vice versa. The circuitry 600 that performs the self-inductance measurements can be included, e.g., in the electronics unit 312 of FIG. 3. The circuitry 600 can drive field generators 602a, 602b (e.g., transmitter coils, field generators described above, etc.) to create electromagnetic fields. For example, the circuitry can include waveform sources 604a, 604b (e.g., individual sources, multiplexed sources, etc.) which can vary amplitude, phase, frequency, waveshape, etc. The circuitry can also include amplifiers 606a, 606b, which can be used to drive the field generators 602a, 602b, respectively. The circuitry includes resistors 608a, 608b, which can be used to measure the current through the respective field generators.

A first switch 610a (e.g., a double pole double throw (DPDT) switch) can be set to position a while driving the first field generator 602a. A second switch 610b (e.g., a DPDT switch) can be set to position b. This allows the generator 602b to act as a sensor, whose signals are amplified by an amplifier 612b. Similarly, the second switch 610b can be set to position a while driving the second field generator 602b, and the first switch 610a can be set to position b, allowing the generator 602a to act as a sensor whose signals are amplified by amplifier 612a. In some implementations, multiple field generators can act as sensors while a generator is being driven.

Once the drive current and induced voltage across a field generator are measured, a mutual inductance matrix M can be determined, e.g., with the following equation, where the voltage induced on coil α due to a transmitting coil β is V_{α,β}:

$${V}_{a , β} ( t ) = {M}_{a , β} \frac{{dI}_{β} ( t )}{dt} \qquad ( 4 )$$

For a number N of field generators, this equation in matrix form is equivalent to:

$$[ \begin{matrix}0 & ⋯ & {V}_{1 , β} \\ ⋮ & ⋱ & ⋮ \\ {V}_{α , 1} & ⋯ & 0\end{matrix} ] = [ \begin{matrix}0 & ⋯ & {M}_{1 , β} \\ ⋮ & ⋱ & ⋮ \\ {M}_{α , 1} & ⋯ & 0\end{matrix} ] [ \begin{matrix}\frac{{dI}_{1} ( t )}{dt} & ⋯ & 0 \\ ⋮ & ⋱ & ⋮ \\ 0 & ⋯ & \frac{{dI}_{N} ( t )}{dt}\end{matrix} ] \qquad ( 5 )$$

As described above, the measurements for drive current and induced voltage can be performed, e.g., by instrumentation. The instrumentation can also determine other values (e.g., derivatives, magnitudes, phases, etc.) by processing the waveforms.

Additionally, some implementations can utilize both self-inductance and mutual-inductance measurements. Utilizing both measurements can yield a full inductance matrix LM. Such combinations can provide additional information for determining and compensating for distortion. For example, combining equations (3) and (5) yields:

$$[ \begin{matrix}{V}_{1 , 1} & ⋯ & {V}_{1 , β} \\ ⋮ & ⋱ & ⋮ \\ {V}_{α , 1} & ⋯ & {V}_{N , N}\end{matrix} ] = [ \begin{matrix}{L}_{1 , 1} & ⋯ & {M}_{1 , β} \\ ⋮ & ⋱ & ⋮ \\ {M}_{α , 1} & ⋯ & {L}_{N , N}\end{matrix} ] [ \begin{matrix}\frac{{dI}_{1} ( t )}{dt} & ⋯ & 0 \\ ⋮ & ⋱ & ⋮ \\ 0 & ⋯ & \frac{{dI}_{N} ( t )}{dt}\end{matrix} ] \qquad ( 6 )$$

This combination can be solved for the LM matrix of both self and mutual inductances. Multiple measurements can be taken, e.g., at arbitrary frequencies, to yield additional data. The measurements and inductances can be processed by a machine learning system to perform distortion compensation, as described further below. In some implementations, sensing devices other than field generators can be used for self and mutual inductances. This can be advantageous, e.g., due to cost or complexity issues associated with switching field generators from transmitting to receiving and vice versa.

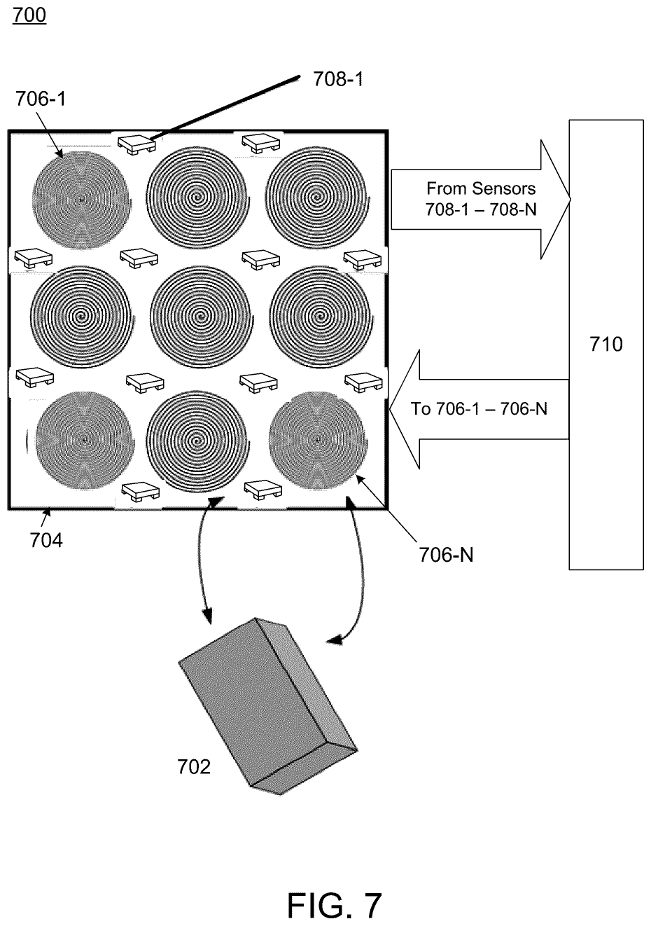

FIG. 7 illustrates another exemplary magnetic tracking system 700 in the presence of a distorter 702 (e.g., similar to distorter 302, referring to FIG. 3) so that the tracking system can compensate for the resulting distortions. The tracking system 700 can be similar to the tracking system 100 of FIG. 1. The tracking system 700 includes a magnetic field generating assembly 704 that is configured to generate magnetic fields within a given a three dimensional region.

The magnetic field generating assembly 704 includes field generators 706-1-706-N that each include one or more electromagnetic coils that produce a magnetic field (e.g., by passing current through each coil). The magnetic field generating assembly 704 can also include any number of sensors 708-1-708-N to measure the inductance of each field generator and the mutual inductances between individual field generators (e.g., coupling measurements). The signals received by the sensors can be transmitted (e.g., through a wire, wirelessly, etc.) to electronics unit 710, which can be similar to the electronics unit 312 of FIG. 3 or 406 of FIG. 4 and can include circuitry similar to the example circuits illustrated in FIGS. 5 and 6 above. The electronics unit 710 can perform amplification, selection, signal processing, digitization, demodulation, etc. of the transmitted signals to prepare the signals for a machine learning model. The electronics unit 710 also drives the magnetic field generating assembly 704.

The sensors 708-1-708-N can include coils, semiconductor devices, Hall Effect sensors, anisotropic magneto-resistive (AMR) sensors, tunneling magneto-resistance (TMR) sensors, giant magnetoresistance (GMR) sensors, etc. The effect of the distorter 702 is not measured by the field generators 706-1-706-N, but is instead measured by the sensors 708-1-708-N. In some implementations, the field generators 706 can be planar while the sensors 708 are multi-axis, or vice versa. In some implementations, the distortion effects of the distorter 702 can be measured by the field generators 706 and the sensors 708.

The methods described above (e.g., by the distortion manager 150) can be used to collect training data for a machine learning model. For example, one or more techniques may be implemented to determine an amount of distortion in a magnetic field. Upon determining the distortive effects of a distorter, these techniques can compensate for the distortion based on provided inductances to a computer system (e.g., the computer system 110). Multiple data sources may be provided to train the machine learning model, such as inductance data of various (e.g., based on shapes, materials, sizes, effects) distorters. As another example, the training data may be provided by collecting self-inductance measurements (e.g., using the circuit of FIG. 5). Additionally, or alternatively, training data can be collected using mutual-inductance measurements (e.g., using the circuit of FIG. 6). Training data may also consist of field measurements from sensors in the tracking volume that are at known positions and orientations, as well as distorted calculations of the position and orientation of sensors in the tracking volume.

FIG. 8 illustrates an exemplary distortion manager 800 that may be stored on or executed by computing device 802 (e.g., server, computer system) and includes a distortion compensator 804. The distortion compensator 804 of the distortion manager 800 includes a data collector 806, which can collect training data such as sets of inductance data 816 that are acquired by sensors. The distortion compensator 804 also includes distortion model trainer 810 to train distortion model 808 by processing extracted training examples from collected data, e.g., inductance data, sensor data. The distortion compensator 804 can include additional submodules, e.g., to perform data filtering and processing, for estimating distortion in an electromagnetic field. The distortion compensator 804 is coupled by the computing device 802 to distortion data, e.g., sensor data 814, inductance data 816, and distorter feature data 818 (e.g., distortion characteristics). In some implementations, the distortion data can be partitioned into any number of ways and can be filtered to include a subset of data, e.g., a portion of the sensor data 814, inductance data 816, and distorter feature data 818. As an example, distorter feature data 818 can include characteristics of distortive effects such as magnetic field strength, shape, direction, etc. These features of the distortion can be learned, saved, and used by the distortion model 808 for training, e.g., to improve characterization of unseen types of distorters and amounts of distortion.

The distortion compensator 804 may operate submodules, e.g., data collector 806, distortion model trainer 810, and distortion model 808, to perform a variety of machine learning tasks, e.g., classification, regression, detection, and estimation. Any submodules of the distortion compensator 804 may be coupled in various configurations to perform the machine learning tasks or to apply artificial intelligence techniques. The distortion compensator 804 may obtain sensor data 814, inductance data 816, and distorter feature data 818 and provide data to the submodules of the distortion compensator 804. For example, the distortion compensator 804 may provide any data type to the data collector 806, to the distortion model 808 for processing and as inputs for estimation, and to be used by the distortion model trainer 810 to train the distortion model 808.

The data collector 806 of the distortion compensator 804 is configured to retrieve data for the distortion manager 800. For example, the data collector 808 may obtain sensor signals, e.g., from a sensor assembly 106 of FIG. 1, to store in sensor data 814. The sensor signals may describe a position, orientation, pose, etc. of sensors in a tracking system environment. The data collector 808 may collect inductance data, e.g., self-inductance measurements, mutual inductance measurements, of a field generating assembly to store in inductance data 816. The data collector 806 also collect additional types of sensor and inductance data, and may perform operations such as data filtering, data formatting, down-sampling etc. to provide computational advantages in cost and efficiency for the distortion manage 800. In some implementations, the data collector 806 may perform interpolation of collected data prior to providing the data when extracting training examples.

The distortion manager 800 may be connected to multiple external storage devices and sources for inductance data, e.g., multiple distortion managers in multiple training and computational environments. In some implementations, inductance data 816 includes previously measured inductances of the field generators absent the presence of a distorter. The inductance data 816 includes previously measured inductances of the field generators in the presence of various types of distorters. In some implementations, the inductance data 816 may be previously stored and retrieved by the distortion compensator 804.

Along with inductance data 816, the data collector 806 may provide the distortion model trainer 810 other types of data from sensors, e.g., data represented by sensor data 814. The sensor data 814 includes signals received, transmitted, and used by the sensor, e.g., signals that can be used to determine position, orientation, pose. The sensor data 814 can include historical data regarding previously estimated or determined positions, orientations, of one or more sensors. In some implementations, sensor data 814 includes previously estimated positions and orientations, e.g., poses, of sensors absent the presence of a distorter. The sensor data 814 can also include previously estimated positions and orientations, e.g., poses, of sensors in the presence of various types of distorters.

As illustrated in FIG. 8, the distortion model trainer 810 may be configured to train the distortion model 808 to identify various features of distorters, e.g., shape, size, strength, material composition. For example, the distortion model trainer 810 may store distorter features (e.g., calculated feature vectors) in distorter feature data 818 for later retrieval and use. Such distorter feature data 818 may be provided from sources other than the distortion model trainer 810. The distorter feature data 818 can include feature vectors representing inputs for estimating the detect distortion. The distortion model 808 may similarly store data (e.g., tensors, mappings, embeddings, encodings) representing distorter features in the distorter feature data 818. In some arrangements, such distorter features may be directly provided to the distortion model trainer 810, the distortion model 808, etc. and correspondingly stored in the distorter feature data 818. In other arrangements, calculations may be executed by the distortion model trainer 810, the distortion model 808, etc. to produce the distorter features prior to being stored in the distorter feature data 812. For example, numerical values representing one or more distorter features (e.g., feature vectors) may be computed from distortion data by the distortion model trainer 810, the distortion model 808, etc. As illustrated in the figure, such stored distorter feature data may reside in the distortion model trainer 810 (e.g., in the distorter feature data 812). Such distorter feature data may be provided to or received from other locations internal or external to the distortion manager 800. For example, the data may be provided for further analysis, storage, etc. to other systems remotely located from the distortion manager 800.

In general, the distortion model trainer 810 may employ one or more techniques to produce the distortion model 808 (e.g., a neural network). For example, the distortion data for may be used to determine an objective function, for which the distortion model 808 is trained to maximize or optimize in some way. The distortion data in the databases can include training examples (e.g., from previous training iterations, current training iterations) in which the distortion model trainer 810 provides to the distortion model 808 for training. The distortion model 808 can learn mappings between the training examples (e.g., sensor measurements, inductance measurements) and determined amounts of distortion. Furthermore, the distortion model 808 can also be trained to map compensations for the determined amounts of distortion, e.g., to improve accuracy of the sensors capturing measurements affected by the detected distortion. In some implementations, the distortion model 808 may be trained to minimize a loss function as a means to estimate distortion, e.g., for a previously unknown/unseen amount of distortion.

A trained distortion model 808 can be used to determine the distortive effects of distorters in a tracking system, e.g., affecting the electromagnetic field of the tracking system. As an example, inductance data may be provided to a trained distortion model 808 to determine the presence of a distorter and/or amount of distortion affecting the field. The trained distortion model 808 can estimate an amount of distortion in the electromagnetic field, and provide a compensation factor, e.g., an adjustment to one or more sensors, for the tracking system. As an example, the trained distortion model 808 may also perform classification to determine a type and severity (e.g., distortive effects) of a distorter in the electromagnetic field. In some implementations, the trained distortion model 808 can predict effects of distorters (e.g., error) on sensor measurements of a tracking system. As an example, the trained distortion model 808 can estimate a position error and an orientation error for one or more sensors of the tracking system, based on the distortion effects of the distorter.

In the illustrated example shown in FIG. 8, the functionality of the data collector 806, the distortion model trainer 810, and the distortion model 808 are presented as being included in the distortion manager 800. However, in some arrangements, the functionality of one or more of these modules may be provided external from the distortion manager 800. Similarly, the simulated sensor database 808, inductance database 810, and distorter feature data 812 are stored in the storage device 808 in this example. However, one or more of these databases may be stored external to the storage device 808 and in some arrangements one or more of the databases may be stored external to the distortion manager 800. In some arrangements, the distortion manager 800 may be implemented in software, hardware, or combinations of hardware and software. Similarly, the modules included in the distortion manager 800 may individually be implemented in hardware and/or software.

The distortion model 808 may perform many training updates based on different training examples provided e.g., collecting inductance data. The distortion model 808 may perform many iterations e.g., millions, of training to gradually and incrementally learn how to make more accurate estimates for distortion. Through the collection of training data from various inductance measurements, the distortion model 808 improve the accuracy of the distortion predictions and estimates over time, learning to accurately estimate and identify features across different distorter types, shapes, sizes, and materials.

In some implementations, the distortion model 808 may perform training to determine how to adjust parameters based on ground truth measurements. For example, the distortion model 808 may identify an estimated distortion and identified type for a distorter in a tracking system. For example, the estimated distortion based on processed inductance data may be compared with a ground truth measurement of distortion for a distorter. If the estimated distortion exceeds a threshold value, e.g., an error value, the distortion model 808 can adjust model parameters and repeat processing until the estimated distortion is within the threshold value from the ground truth distortion. In another example, if the identified severity of the distorter is in the wrong classified category, e.g., incorrectly identifying a severity of the distortion, then the distortion model 808 can identify certain features of the distorter and repeat training until the correct severity label is determined for the distorter, e.g., adjusting parameters. An adjustment of the distortion model 808 may include adjusting the values of weights and biases for nodes in one or more neural networks.

In some implementations, the distortion model 808 may adjust a penalty parameter. In some implementations, parameters adjusted in distortion model 808 can be learned e.g., by a neural network that can include the distortion model 808. In some implementations, model parameters adjusted for the distortion model 808 can include coefficients or weights of a neural network, biases of a neural network, and cluster centroids in clustering networks. In some implementations, hyperparameters e.g., parameters to adjust learning of the distortion model 808, can be adjusted for training the distortion model 808. Hyperparameters may include a test-train split ratio, learning rates, selection of optimization algorithms, selection of functions e.g., activation, cost, or loss functions, a number of hidden layers, a dropout rate, a number of iterations, a number of clusters, a pooling size, a batch size, and a kernel or filter size in convolutional layers.

The distortion model 808 can use any appropriate algorithm such as backpropagation of error or stochastic gradient descent for training. Through many different training iterations, based on training data and examples provided to the distortion model 808, the distortion model 808 learns to accurately estimate distortion. The distortion model 808 can be trained on time-series inductance data over a time period e.g., hours, days, weeks, and so on. The distortion model 808 is evaluated for error and accuracy over a validation set. The model training continues until either a timeout occurs, e.g., typically several hours, or a predetermined error or accuracy threshold is reached. In some implementations, an ensemble approach of models may be implemented by the distortion model 808 to improve overall accuracy of estimated distortion. Model training and re-training of the distortion model 808 can be performed repeatedly at a pre-configured cadence e.g., once a week, once a month, and if new data is available in the object store then it automatically gets used as part of the training. The data pipeline to obtain new data remains the same as described above.

In some implementations, the distortion model 808 can include feed-forward neural networks with multiple feed-forward layers. Each feed-forward neural network can include multiple fully-connected layers, in which each fully-connected layer applies an affine transformation to the input to the layer, i.e., multiplies an input vector to the layer by a weight matrix of the layer. Optionally, one or more of the fully-connected layers can apply a non-linear activation function e.g., ReLU, logistic, hyperbolic tangent, to the output of the affine transformation to generate the output of the layer. In some implementations, the distortion model 808 can include regression e.g., linear, logistic, polynomial, ridge, LASSO techniques.

The distortion model 808 can perform of variety of training techniques to improve distortion estimation of distorters in a tracking system environment, including supervised and unsupervised learning. In some examples, the distortion model 808 performs hybrid-learning techniques to improve distortion estimation. The training of the distortion model 808 can be performed using obtained ground truth data that includes known distorters, coupled with inductance measurements of the known distorters. The distortion model 808 can adjust one or more weights or parameters to match estimates or predictions from the distortion model 808 to the ground truth data. In some implementations, the distortion model 808 includes one or more fully or partially connected layers. Each of the layers can include one or more parameter values indicating an output of the layers. The layers of the distortion model 808 can generate distortion estimates for distorters in a tracking system environment, which can be used to perform one or more control actions in the tracking system environment, e.g., adjusting the estimated position of one or more sensors, medical device, etc.

Along with providing needed distortion information, the distortion manager 800 may perform other functions, e.g., distorter training, distorter testing, and validation. The distortion manager 800 may also prepare distortion estimates in advance of planned use of a tracking system, e.g., electromagnetic tracking system 100. For example, as new distorters are placed in the presence of the tracking system, the distortion manager 800 may categorize the resulting distortions and determine similarities with these distortions and previously measured distortions. The distortion manager 800 enables improved efficiency in in providing distortion information regarding a new, e.g., unknown, distorter to the computer system 110 and other potential recipient devices.

Using artificial intelligence techniques can provide the distortion model with numerous approaches in training, e.g., unsupervised learning, supervised learning, and so on, to estimate an accurate distortion amount. Additionally, the techniques for the distortion model may be performed in various implementations and configurations, e.g., an offline mode to train the model while inductance and sensor data is collected. By applying artificial intelligence and machine learning techniques, the distortion model 808 of the distortion manager 800 determines effects of field distorting objects, e.g., distorters, in a generated magnetic field. The distortion effects may be quantified as an amount of distortion in magnetic field strength units, e.g., in Tesla, Gauss, etc. identified in the generated magnetic field, but may also be described by a type, e.g., based on size, material composition, and so on. In some implementations, the distortion effects may be quantified in relation to the expected magnetic field strength, e.g., a quantity of the field degraded, mitigated, or affected by the distortion effects.

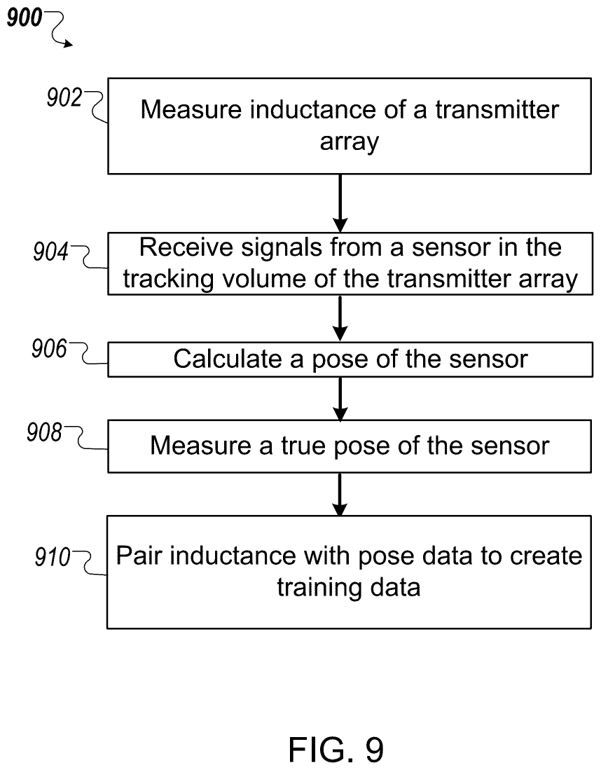

FIG. 9 is a flowchart of an exemplary process 900 to collect training data without the presence of distorters to train a distortion compensation machine learning system. For example, the distortion manager 150 of FIG. 1 can perform the process in order to train a distortion model trainer, by using a data collector to extract or generate training examples. Collecting training data without the presence of distorters can provide baseline data for the tracked location of a sensor without distortions in the magnetic field interfering with transmitted and received signals.

The inductance of the transmitter array is measured (902), e.g., through self-inductance measurements, mutual-inductance measurements, etc. The inductance of the transmitter array can be stored, e.g., as inductance data 816 (referring to FIG. 8 above.) As an example, self-inductance measurements can be collected using the circuit of FIG. 5 (shown above). Additionally, or alternatively, mutual-inductance measurements can be collected using the circuit of FIG. 6 (shown above). The inductance measurements can be provided to the data collector, to be retrieved or accessed by the distortion model trainer.

The data collector of a distortion compensation machine learning system is configured to receive signals provided by a sensor placed within a tracking volume of the transmitter array (904). The data collector may receive signals from a sensor, in which the received signals provide measurements representing the pose of the sensor. In some implementations, the received signals represent time-series data capturing position, velocity, acceleration (e.g., location and movement) of the sensor. In some examples, multiple sensors placed within the tracking volume of the transmitter array provide a single, or multiple signals representing pose measurements.

The computing device of the distortion compensation machine learning system calculates a pose, e.g., position and orientation data of the sensor based on the received signals (906). For example, the received signals include measurements corresponding to the orientation and position to compute the pose of the sensor. In some implementations, a time series of the sensor's pose may be computed. In some examples, the pose of multiple sensors may be computed.

A true pose, e.g., measured pose data, of a sensor is measured and compared with the calculated pose of the sensor (908). For example, the true pose of the sensor and the calculated pose of the sensor may demonstrate a significant error, e.g., exceeding a tolerance value. The true pose of the sensor is measured to re-calibrate the distortion compensation machine learning system if the calculated pose of the sensor exceeds the allowed tolerance value.

The inductance data and true pose of a sensor are paired to create a training data (910). The data collector and the distortion model trainer of the distortion compensation machine learning system can extract training examples, e.g., pairs of inductance data and pose data. In some implementations, the inductance data, true pose data, and calculated pose data may be coupled together to generate a training example.

By generating a baseline training example, the distortion model of the distortion compensation machine learning system may be trained to determine absence of a distorter when the tracking system is used without a distorter. For example, the distortion model may estimate an insignificant amount of distortion (e.g., less than a threshold value) indicating an absence of a distorter. In some examples, a categorical label indicating the absence of a distorter may be generated as an output of the distortion model.

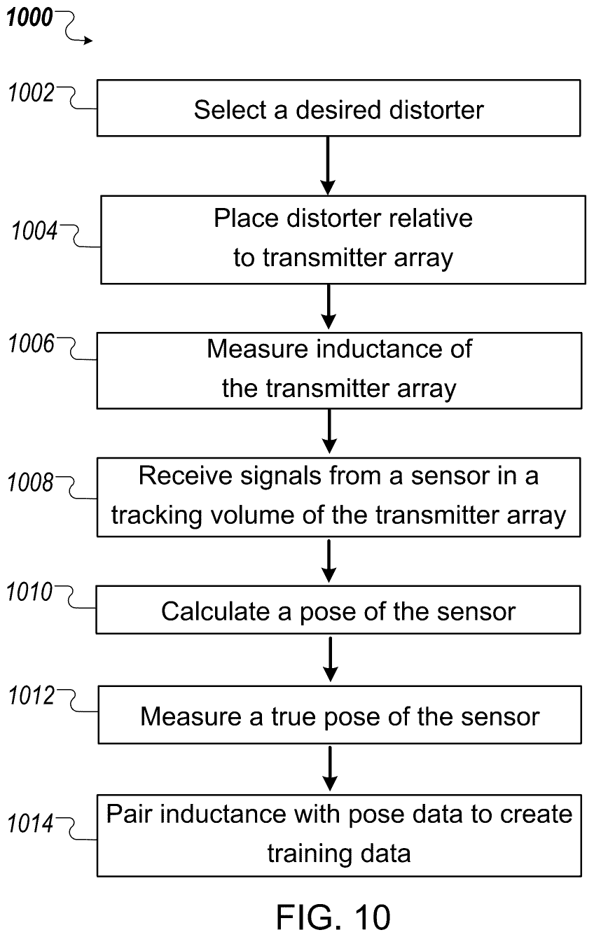

FIG. 10 is a flowchart of an exemplary process 1000 to collect training data in the presence of distorters to train a distortion compensation machine learning system. For example, the distortion manager 150 of FIG. 1 can perform the process in order to train a distortion model trainer, by using a data collector to extract or generate training examples. Collecting training data in the presence of a variety of distorters can improve the accuracy of the distortion model.

A distorter is selected to be placed in proximity to the tracking system, e.g., based on the material, shape, size (1002). For example, an example tracking system may use multiple sensors to provide guidance, e.g., tracking assistance of medical devices, tools, and so on, for medical procedures. As discussed, distorters can be made of any material that affects magnetic field intensity and/or shape. Various shapes and sizes may be used as a distorter to generate training examples.

The distorter can then be placed in proximity to the transmitter array (1004). For example, the distorter can be placed below, above, etc. the transmitter array based on the surrounding environment of the tracking system. In some implementations, the distorter may be moved to multiple positions relative to the transmitter array. A distorter may start at one position relative to the transmitter and adjust to a second position, third position, etc. while steps of process 1000, e.g., 1006, 1008, are performed. A training example generated by process 1000 can include placing the distortion at multiple positions represented at multiple time indices (e.g., time series data).

The inductances of the transmitter array are measured (e.g., through self-inductance measurements, mutual-inductance measurements, sensor signals, etc.) while the transmitter array is in the presence of the distorter (1006). The inductance measurements may be collected self-inductance measurements (e.g., using the circuit of FIG. 5) as well as collected mutual-inductance measurements (e.g., using the circuit of FIG. 6). The inductance measurements of the transmitter array can be stored, e.g., in a database, e.g., inductance data 814 of FIG. 8, described above.

A sensor can be placed within a tracking volume of the transmitter array, and the distortion manager 150 receives signals from the sensor (1008). For example, the received signals include measurements corresponding to the orientation, location, and other aspects of the sensor's pose. In some implementations, a time series of the sensor's pose may be measured. In some examples, multiple sensors may be placed within the tracking volume.

The received signals can be used to calculate a pose (e.g., orientation, location, etc.) of the sensor (1010). For example, the computing device of the distortion compensation machine learning system calculates a pose, e.g., pose data of the sensor based on the received signals. The orientation, location, and other aspects of the sensor's position to compute the pose of the sensor. In some implementations, a time series of the sensor's pose may be computed. In some examples, the pose of multiple sensors may be computed. The computations for sensor position, orientation, and therefore pose may include applications of physical model equations.

The true pose of the sensor can be measured (1012). For example, the previously calculated pose is likely to be different than the true pose of the sensor because of the distortions in the magnetic field. The true pose of the sensor may be measured by removing one or more sources of distortion from the tracking system to obtain an accurate measurement of true pose, e.g., true pose data, for the sensor. In some implementations, a true pose may be measure for multiple sensors in the tracking system. In some implementations, the true pose can be provided by camera devices, e.g., optical system 130, referring to FIG. 1 above.

The inductance data and true pose of a sensor are paired to create a training example, e.g., training data (1014). The data collector and the distortion model trainer of the distortion compensation machine learning system can extract training examples, e.g., pairs of inductance data and pose data. In some implementations, the inductance data, true pose data, and calculated pose data may be coupled to generate a training example.

The distortion model, once trained, can compensate for the distortions in the magnetic field and calculate the true pose of the sensor from the received signals.

By generating a distortion-based training example, the distortion model of the distortion compensation machine learning system may be trained to determine an amount of distortion generated by one or more distorters in proximity to the tracking system. For example, the distortion model may estimate a significant amount of distortion (e.g., greater than a threshold value) and compensate for the distortion. In some examples, a categorical label indicating the presence and/or severity (e.g., low, medium, high, severe) of a distorter may be generated. In some implementations, the distortion compensation machine learning system may be trained to determine distortion impact of one or more known distorters, e.g., an estimate of the effect of a known distorter in the tracking system.

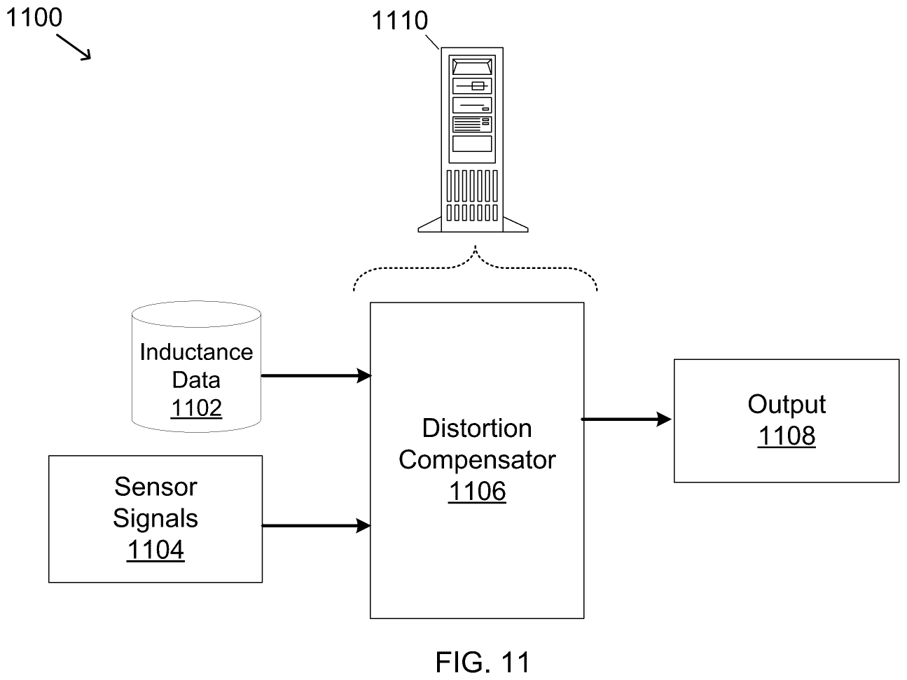

FIG. 11 is a data flow for a process 1100 representing operation of a distortion model, e.g., distortion model 808 machine after being trained by distortion model trainer 810. As described above, training examples, e.g., inductance measurements and at least one of true pose data or calculated pose data, can be extracted from the training data.

The training examples may be extracted from inductance data obtained in a tracking system absent any distorters, e.g., such as data collected from a process such as process 900 shown in FIG. 9). The training examples may also be extract from inductance data obtained in a tracking system data with any number of distorters, e.g., such as data collected from a method such as process 1000 shown in FIG. 10. In some implementations, training examples may be extracted that include inductance data corresponding to the presence of distorters in some time periods and the absence of distorters in other time periods.

One or more sets of inductance data 1102 (e.g., received inductances from a transmitter array) and sensor signals 1104 (e.g., received from a sensor within the tracking volume of the transmitter array) can be provided to the distortion compensator 1106. The distortion compensator 1106 can determine a pose of the sensor from the received inductance data 1102 and the received sensor signals 1104. The distortion compensator 1106 generates an output 1108 representing the true pose of one or more corresponding sensors. The true pose of the one or more corresponding sensors relate to the input data, e.g., one or more sets of inductance data 1102 and one or more sets of sensor signals 1104 provided.

The distortion compensator 1106 may determine a number of true poses corresponding to a number of sensors, based on the processed data inputs. For example, the distortion compensator 1106 can determine a respective compensation for each sensor to improve the estimate of the true pose of the respective sensor. By improving accuracy of true pose estimations, the sensor measurement accuracy can be compensated to account for amount of distortion generated by a distorter. The true pose data can be represented in any form, e.g., various coordinate systems, and in some implementations, may include a confidence score based on the training examples that the distortion compensator 1106 was able to extract. For example, a confidence score or level may be assigned to each true pose outputted by the distortion compensator 1106 to indicate a likelihood that the true pose is correct, e.g., within a threshold value, assigned a correct labeled.

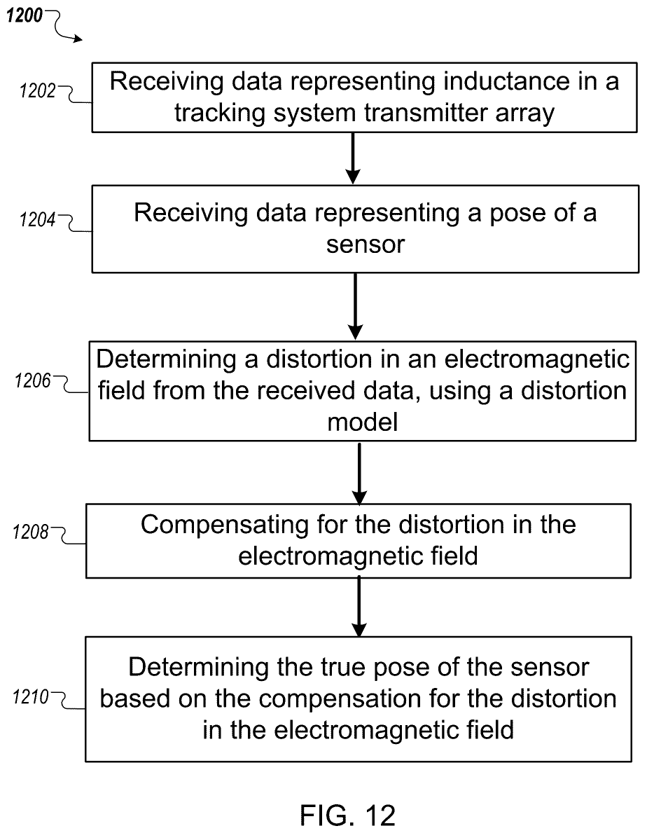

FIG. 12 is a flowchart of a process 1200 representing operation of a distortion manager to compensate for distortion in a tracking system. For example, the process 1200 can be performed by the distortion manager 150 of FIG. 1.

The distortion manager receives data representing inductance in a tracking system transmitter array that can generate an electromagnetic field (1202). For example, the distortion manager may receive inductance data that is collected using self-inductance measurements and mutual-inductance measurements of the electromagnetic field, as described in FIGS. 5 and 6 respectively.

The distortion manager receives data representing a pose, e.g., a position and orientation of a sensor (1204). In some implementations, the distortion manager receives data representing multiple poses of a sensor, a pose for each sensor in a set of sensors, multiple poses for multiple sensors, or some combination therein. The distortion manager may receive signals from a sensor or a computer coupled to the sensor that indicate the sensor's pose, including the position and orientation of the sensor.

The distortion manager determines a distortion in the electromagnetic field of the tracking system transmitter array, from the received inductance data (1206). For example, the distortion manager can determine the distortion by using a distortion model, e.g., distortion model 808 (referring to FIG. 8). A distortion model trainer, e.g., distortion model trainer 810, may employ artificial intelligence techniques, machine-learning algorithms, or some combination therein to train and test the distortion model using the inductance data. The distortion model trainer of a distortion model may compare the estimated distortion to a ground truth measurement of distortion in the electromagnetic field. As an example, the estimated distortion may be exceed a threshold error value, in which the distortion model trainer repeats training of the distortion model until the distortion model generates estimated distortion values with the threshold error value. In some implementations, the threshold error value is a learnable parameter that can be trained and updated by the distortion model trainer and the distortion model.

The distortion model uses training examples that include receiving a first set of inductance data representing inductance measurements of the electromagnetic field without a distorter in the transmitter array. The training example also includes a second set of inductance data representing inductance measurements of the electromagnetic field with one or more distorters in the transmitter array. The distortion model can process both the first set of inductance data and the second set of inductance data to identify patterns in distortion detection and estimation. The distortion model may also compares both the first set of inductance data and the second set of inductance data to optimize an underlying objective function that estimates the amount of distortion detected in the electromagnetic field of the transmitter array.

The distortion manager determines a compensation for sensors based on the distortion in the electromagnetic field of the transmitter array (1208). For example, the distortion manager may use a generated or determined value describing an amount of distortion detected in the electromagnetic field. The amount of distortion detected may be referred to as a compensation value, an adjustment factor, etc. The distortion manager determines a series of calibrations or correction factors across different dimensions, e.g., degrees of freedom, for a sensor based on pose, e.g., position and orientation. By doing so, the distortion manager determines an exact mapping for measurements from a sensor, or multiple sensors, adjusted to include compensation from distortion and to identify a true estimate of pose, e.g., a true pose of one or more sensors.

The distortion manager determines the true pose, e.g., position and orientation of the sensor (or multiple sensors) based on the compensation for the distortion in the electromagnetic field (1210). The distortion manager applies the determined distortion compensation to accurately determine a position and orientation for each sensor in a transmitter array. The updated values for the true pose of a sensor or multiple sensors may be provided to a computing device. In some implementations, the true pose may be provided for display, as well as transmitted to a remote device or server. In some implementations, the true pose may be continually updated and provided as a time-series of estimates to record or track the one or more sensors in an environment.

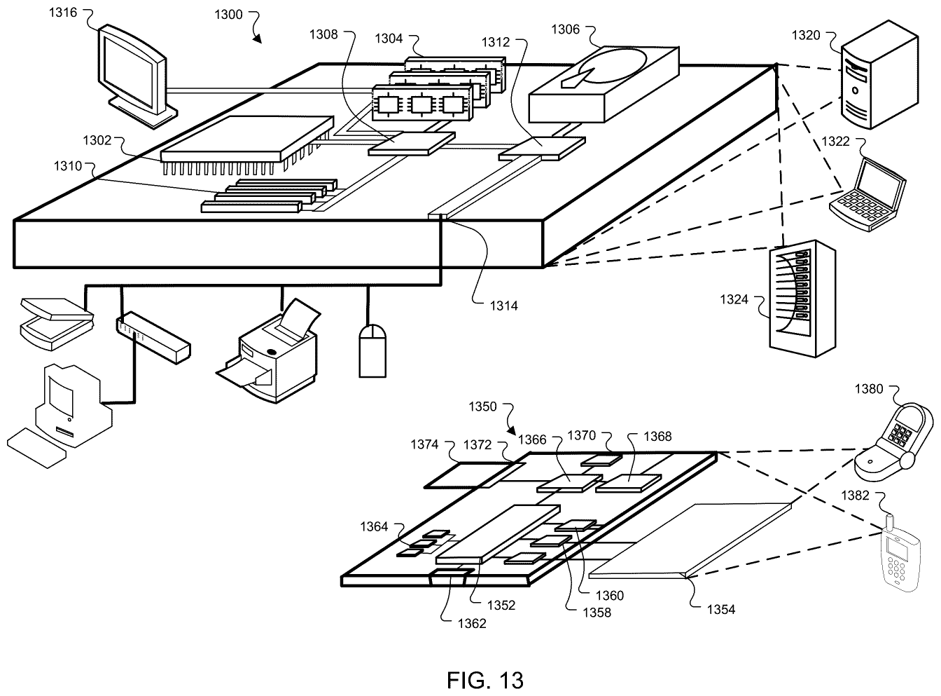

FIG. 13 shows an example computing device 1300 and an example mobile computing device 1350, which can be used to implement the techniques described herein. For example, the computing device 1300 may be implemented as the computer system 110 of FIG. 1. Computing device 1300 is intended to represent various forms of digital computers, including, e.g., laptops, desktops, workstations, personal digital assistants, servers, blade servers, mainframes, and other appropriate computers. Computing device 1350 is intended to represent various forms of mobile devices, including, e.g., personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 1300 includes processor 1302, memory 1304, storage device 1306, high-speed interface 1308 connecting to memory 1304 and high-speed expansion ports 1310, and low speed interface 1312 connecting to low speed bus 1314 and storage device 1306. Each of components 1302, 1304, 1306, 1308, 1310, and 1312, are interconnected using various busses, and can be mounted on a common motherboard or in other manners as appropriate. Processor 1302 can process instructions for execution within computing device 1300, including instructions stored in memory 1304 or on storage device 1306, to display graphical data for a GUI on an external input/output device, including, e.g., display 1316 coupled to high-speed interface 1308. In some implementations, multiple processors and/or multiple buses can be used, as appropriate, along with multiple memories and types of memory. In addition, multiple computing devices 1300 can be connected, with each device providing portions of the necessary operations (e.g., as a server bank, a group of blade servers, a multi-processor system, etc.).

Memory 1304 stores data within computing device 1300. In some implementations, memory 1304 is a volatile memory unit or units. In some implementation, memory 1304 is a non-volatile memory unit or units. Memory 1304 also can be another form of computer-readable medium, including, e.g., a magnetic or optical disk.

Storage device 1306 is capable of providing mass storage for computing device 1300. In some implementations, storage device 1306 can be or contain a computer-readable medium, including, e.g., a floppy disk device, a hard disk device, an optical disk device, a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, including, e.g., those described above. The data carrier is a computer- or machine-readable medium, including, e.g., memory 1304, storage device 1306, memory on processor 1302, and the like.

High-speed controller 1308 manages bandwidth-intensive operations for computing device 1300, while low speed controller 1312 manages lower bandwidth-intensive operations. Such allocation of functions is an example only. In some implementations, high-speed controller 1308 is coupled to memory 1304, display 1316 (e.g., through a graphics processor or accelerator), and to high-speed expansion ports 1310, which can accept various expansion cards (not shown). In some implementations, the low-speed controller 1312 is coupled to storage device 1306 and low-speed expansion port 1314. The low-speed expansion port, which can include various communication ports (e.g., USB, Bluetooth®, Ethernet, wireless Ethernet), can be coupled to one or more input/output devices, including, e.g., a keyboard, a pointing device, a scanner, or a networking device including, e.g., a switch or router (e.g., through a network adapter).

Computing device 1300 can be implemented in a number of different forms, as shown in FIG. 13. For example, the computing device 1300 can be implemented as standard server 1320, or multiple times in a group of such servers. The computing device 1300 can also can be implemented as part of rack server system 1324. In addition or as an alternative, the computing device 1300 can be implemented in a personal computer (e.g., laptop computer 1322). In some examples, components from computing device 1300 can be combined with other components in a mobile device (e.g., the mobile computing device 1350). Each of such devices can contain one or more of computing device 1300, 1350, and an entire system can be made up of multiple computing devices 1300, 1350 communicating with each other.

Computing device 1350 includes processor 1352, memory 1364, and an input/output device including, e.g., display 1354, communication interface 1366, and transceiver 1368, among other components. Device 1350 also can be provided with a storage device, including, e.g., a microdrive or other device, to provide additional storage. Components 1350, 1352, 1364, 1354, 1366, and 1368, may each be interconnected using various buses, and several of the components can be mounted on a common motherboard or in other manners as appropriate.

Processor 1352 can execute instructions within computing device 1350, including instructions stored in memory 1364. The processor 1352 can be implemented as a chipset of chips that include separate and multiple analog and digital processors. The processor 1352 can provide, for example, for the coordination of the other components of device 1350, including, e.g., control of user interfaces, applications run by device 1350, and wireless communication by device 1350.

Processor 1352 can communicate with a user through control interface 1358 and display interface 1356 coupled to display 1354. Display 1354 can be, for example, a TFT LCD (Thin-Film-Transistor Liquid Crystal Display) or an OLED (Organic Light Emitting Diode) display, or other appropriate display technology. Display interface 1356 can comprise appropriate circuitry for driving display 1354 to present graphical and other data to a user. Control interface 1358 can receive commands from a user and convert them for submission to processor 1352. In addition, external interface 1362 can communicate with processor 1342, so as to enable near area communication of device 1350 with other devices. External interface 1362 can provide, for example, for wired communication in some implementations, or for wireless communication in some implementations. Multiple interfaces also can be used.

Memory 1364 stores data within computing device 1350. Memory 1364 can be implemented as one or more of a computer-readable medium or media, a volatile memory unit or units, or a non-volatile memory unit or units. Expansion memory 1374 also can be provided and connected to device 1350 through expansion interface 1372, which can include, for example, a SIMM (Single In Line Memory Module) card interface. Such expansion memory 1374 can provide extra storage space for device 1350, and/or may store applications or other data for device 1350. Specifically, expansion memory 1374 can also include instructions to carry out or supplement the processes described above and can include secure data. Thus, for example, expansion memory 1374 can be provided as a security module for device 1350 and can be programmed with instructions that permit secure use of device 1350. In addition, secure applications can be provided through the SIMM cards, along with additional data, including, e.g., placing identifying data on the SIMM card in a non-hackable manner.

The memory 1364 can include, for example, flash memory and/or NVRAM memory, as discussed below. In some implementations, a computer program product is tangibly embodied in a data carrier. The computer program product contains instructions that, when executed, perform one or more methods. The data carrier is a computer- or machine-readable medium, including, e.g., memory 1364, expansion memory 1374, and/or memory on processor 1352, which can be received, for example, over transceiver 1368 or external interface 1362.

Device 1350 can communicate wirelessly through communication interface 1366, which can include digital signal processing circuitry where necessary. Communication interface 1366 can provide for communications under various modes or protocols, including, e.g., GSM voice calls, SMS, EMS, or MMS messaging, CDMA, TDMA, PDC, WCDMA, CDMA2000, or GPRS, among others. Such communication can occur, for example, through radio-frequency transceiver 1368. In addition, short-range communication can occur, including, e.g., using a Bluetooth®, WiFi, or other such transceiver (not shown). In addition, GPS (Global Positioning System) receiver module 1370 can provide additional navigation- and location-related wireless data to device 1350, which can be used as appropriate by applications running on device 1350.

Device 1350 also can communicate audibly using audio codec 1360, which can receive spoken data from a user and convert it to usable digital data. Audio codec 1360 can likewise generate audible sound for a user, including, e.g., through a speaker, e.g., in a handset of device 1350. Such sound can include sound from voice telephone calls, recorded sound (e.g., voice messages, music files, and the like) and also sound generated by applications operating on device 1350.

Computing device 1350 can be implemented in a number of different forms, as shown in FIG. 13. For example, the computing device 1350 can be implemented as cellular telephone 1380. The computing device 1350 also can be implemented as part of smartphone 1382, personal digital assistant, or other similar mobile device.

Various implementations of the systems and techniques described here can be realized in digital electronic circuitry, integrated circuitry, specially designed ASICS (application specific integrated circuits), computer hardware, firmware, software, and/or combinations thereof. These various implementations can include one or more computer programs that are executable and/or interpretable on a programmable system. This includes at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described herein can be implemented on a computer having a display device (e.g., a CRT (cathode ray tube) or LCD (liquid crystal display) monitor) for presenting data to the user, and a keyboard and a pointing device (e.g., a mouse or a trackball) by which the user can provide input to the computer. Other kinds of devices can be used to provide for interaction with a user as well. For example, feedback provided to the user can be a form of sensory feedback (e.g., visual feedback, auditory feedback, or tactile feedback). Input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such backend, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the components described herein can be separated, combined or incorporated into a single or combined component. The components depicted in the figures are not intended to limit the systems described herein to the software architectures shown in the figures.

A number of embodiments have been described. Nevertheless, it will be understood that various modifications may be made without departing from the spirit and scope of the disclosure. Accordingly, other embodiments are within the scope of the following claims.

What is claimed is:

1. A computer-implemented method comprising:
receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field;
training a machine learning system for compensating for distortion by using the first set of inductance data and the second set of inductance data, wherein the machine learning system is configured to generate an estimated value of distortion, the estimated value of distortion indicating an amount of distortion present in the electromagnetic field;
receiving an additional set of inductance data representing one or more inductance measurements from the one or more transmitters;
generating, by the trained machine learning system, the estimated value of distortion in the electromagnetic field using the additional set of inductance data and measured pose data, wherein the measured pose data represents position and orientation information for one or more sensors; and
providing the estimated value of the distortion for application to a computing device for calibrating the one or more sensors by compensating for the estimated value of the distortion.

2. The computer-implemented method of claim 1, further comprising:
producing a compensation for at least one sensor of the one or more sensors, based on the estimated value of distortion and the measured pose data; and
applying the compensation to adjust at least one of (i) a position, or (ii) an orientation, for the at least one sensor of the one or more sensors.

3. The computer-implemented method of claim 2, wherein producing the compensation for the at least one sensor of the one or more sensors comprises determining at least one of (i) a first position, or (ii) a first orientation of the at least one sensor based on the measured pose data.

4. The computer-implemented method of claim 3, wherein the first position represents a position of the at least one sensor with the distorter being present and the first orientation represents an orientation of the at least one sensor with the distorter being present.

5. The computer-implemented method of claim 3, wherein producing a compensation for at least one sensor of the one or more sensors comprises determining at least one of (i) a calibrated position, or (ii) a calibrated orientation, for the at least one sensor based on the estimated value of distortion and the measured pose data.

6. The computer-implemented method of claim 5, wherein applying the compensation comprises providing a signal, wherein the signal adjusts the at least one sensor from the first position to the calibration position and the first orientation to the calibration orientation.

7. The computer-implemented method of claim 1, further comprising:
comparing the estimated value of distortion to a threshold value, wherein the distorter is present if the estimated value of distortion exceeds the threshold value.

8. The computer-implemented method of claim 1, wherein the trained machine learning system is configured to generate a distorter output, the distorter output representing at least one of (i) a size, (ii) a type, (iii) a material composition, (iv) a magnetic field shape, or (v) a shape, of the distorter.

9. The computer-implemented method of claim 1, wherein training the machine learning system comprises executing training iterations until an output of the machine learning system satisfies a threshold error.

10. The computer-implemented method of claim 1, wherein training the machine learning system comprises performing training iterations until an output label of the machine learning system substantially matches a predefined distorter label.

11. A system comprising:
one or more computers; and
one or more computer memory devices interoperably coupled with the one or more computers and having tangible, non-transitory, machine-readable media storing one or more instructions that, when executed by the one or more computers, perform one or more operations comprising:
receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field;
training a machine learning system for compensating for distortion by using the first set of inductance data and the second set of inductance data, wherein the machine learning system is configured to generate an estimated value of distortion, the estimated value of distortion indicating an amount of distortion present in the electromagnetic field;
receiving an additional set of inductance data representing one or more inductance measurements from the one or more transmitters;
generating, by the trained machine learning system, the estimated value of distortion in the electromagnetic field using the additional set of inductance data and measured pose data, wherein the measured pose data represents position and orientation information for one or more sensors; and
providing the estimated value of the distortion for application to a computing device for calibrating the one or more sensors by compensating for the estimated value of the distortion.

12. The system of claim 11, further comprising:
producing a compensation for at least one sensor of the one or more sensors, based on the estimated value of distortion and the measured pose data; and
applying the compensation to adjust at least one of (i) a position, or (ii) an orientation, for the at least one sensor of the one or more sensors.

13. The system of claim 12, wherein producing the compensation for the at least one sensor of the one or more sensors comprises determining at least one of (i) a first position, or (ii) a first orientation of the at least one sensor based on the measured pose data.

14. The system of claim 13, wherein the first position represents a position of the at least one sensor with the distorter being present and the first orientation represents an orientation of the at least one sensor with the distorter being present.

15. The system of claim 14, wherein producing a compensation for at least one sensor of the one or more sensors comprises determining at least one of (i) a calibrated position, or (ii) a calibrated orientation, for the at least one sensor based on the estimated value of distortion and the measured pose data.

16. The system of claim 15, wherein applying the compensation comprises providing a signal, wherein the signal adjusts the at least one sensor from the first position to the calibration position and the first orientation to the calibration orientation.

17. The system of claim 11, further comprising:
comparing the estimated value of distortion to a threshold value, wherein the distorter is present if the estimated value of distortion exceeds the threshold value.

18. The system of claim 11, wherein the trained machine learning system is configured to generate a distorter output, the distorter output representing at least one of (i) a size, (ii) a type, (iii) a material composition, (iv) a magnetic field shape, or (v) a shape, of the distorter.

19. The system of claim 11, wherein training the machine learning system comprises executing training iterations until an output of the machine learning system satisfies a threshold error.

20. A non-transitory, computer-readable medium storing one or more instructions executable by a computer system to perform operations comprising:
receiving a first set of inductance data representing one or more inductance measurements when a distorter is absent, and receiving a second set of inductance data representing one or more inductance measurements when the distorter is present, wherein the first set of inductance data and the second set of inductance data is received from an array of electromagnetic transmitters configured to generate an electromagnetic field;
training a machine learning system for compensating for distortion by using the first set of inductance data and the second set of inductance data, wherein the machine learning system is configured to generate an estimated value of distortion, the estimated value of distortion indicating an amount of distortion present in the electromagnetic field;
receiving an additional set of inductance data representing one or more inductance measurements from the one or more transmitters;
generating, by the trained machine learning system, the estimated value of distortion in the electromagnetic field using the additional set of inductance data and measured pose data, wherein the measured pose data represents position and orientation information for one or more sensors; and
providing the estimated value of the distortion for application to a computing device for calibrating the one or more sensors by compensating for the estimated value of the distortion.
